(19) United States
(12) Patent Application Publication
(10) Pub. No.: US 2008/0044740 A1
(43) Pub. Date: Feb. 21, 2008

(54) PHOTOMASK HAVING HAZE REDUCTION LAYER

(75) Inventors: Jiin-Hong Lin, Jhubei City (TW); Chin-Wang Hu, Jhubei City (TW); Ming-Tao Ho, Hsin-Chu City (TW)

Correspondence Address: David M. O'Dell;Attorney for Applicants, Haynes and Boone, LLP, 901 Main Street, Suite 3100, Dallas, TX, 75202-3789, US

(73) Assignee: TAIWAN SEMICONDUCTOR MANUFACTURING COMPANY, LTD., Hsin-Chu (TW)

(21) Appl. No.: 11/466,002

(22) Filed: Aug. 21, 2006

(51) Int. Cl. C23C 14/00; G21G 5/00; A61N 5/00; C23C 14/32; G03F 1/00

(52) U.S. Cl. 430/5; 204/192.1; 250/492.2

(57) ABSTRACT

A photomask is fabricated to have a patterned surface and a transparent layer formed on the patterned surface.

### BACKGROUND

The present disclosure relates in general to semiconductor manufacturing technology, and more particularly, to reducing exposure of a patterned surface of a photomask to a cleaning solution during cleaning of the photomask.

Photomasks, or reticles, are commonly used for photolithography in semiconductor manufacturing. Photomasks are typically made from very flat pieces of quartz or glass with a layer of chromium deposited on one side. The chromium layer is patterned, the pattern being used to transfer an image to a wafer during photolithography processing. While contamination of photomasks has always been a concern, high precision masks, such as those used in photolithography having wavelengths equal to or less than 248 nm, are particularly susceptible to defects.

One type of photomask contamination is referred to as haze contamination. Haze contamination is a precipitant formed during mask manufacture, inspection, and lithography. Generally, chemical contaminants are left on the patterned surface of a photomask during fabrication of the photomask. When that chemical residue is exposed, such as to ultraviolet (UV) light, the chemical residue is sublimated. However, following exposure of the photomask, a residue layer is deposited across the patterned surface of the photomask. This residue layer or “haze” generally builds up slowly, but nevertheless, requires cleaning of the photomask following several photoexposures. As a given photomask is often repeatedly used, during the life of the given photomask, it will be subjected to multiple cleanings. The repeated cleanings can negatively affect the durability of the photomask as well as performance parameters, such as phase angle and transmittance. One proposed solution involves formation of an oxidation layer on the patterned surface of the photomask. It has been found that this oxidation layer, which is generated prior to cleaning of the photomask by exposing the photomask to ultraviolet (UV) light, enhances, albeit slightly, the durability of the photomask.

Formation of the oxidation layer requires relatively strict adherence to control parameters, such as exposure time, reaction temperature, and pressure, to achieve a uniform layer. Furthermore, the phase angle and transmittance of the photomask change following only a few cleanings notwithstanding formation of the oxidation layer. Moreover, the transmittance of the oxidation layer is different from the transmittance of the photomask. After a sufficient number of cleanings, the phase angle and/or transmittance may fall outside an accepted tolerance thereby rendering continued use of the photomask unadvisable.

Therefore, a need exists for a method and an apparatus that reduces haze formation.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. It is also emphasized that the drawings appended illustrate only typical embodiments of this invention and are therefore not to be considered limiting in scope, for the invention may apply equally well to other embodiments.

FIG. 1 is a cross-sectional diagrammatic view of a binary photomask according to one aspect of the present disclosure.

FIG. 2 is a cross-sectional diagrammatic view of a phase shift photomask (PSM) according to one aspect of the present disclosure.

FIG. 3 is a histogram showing variance of phase angle shift of a 248 nm PSM coated with a 500 Å silica protective layer compared to a conventional 248 nm PSM.

FIG. 4 is a histogram showing variance of transmittance for a 248 nm PSM having a 500 Å silica protective layer compared to a conventional 248 nm PSM.

### DETAILED DESCRIPTION

It is to be understood that the following disclosure provides different embodiments, or examples, for implementing different features of the invention. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not itself dictate a relationship between various embodiments and/or configurations discussed.

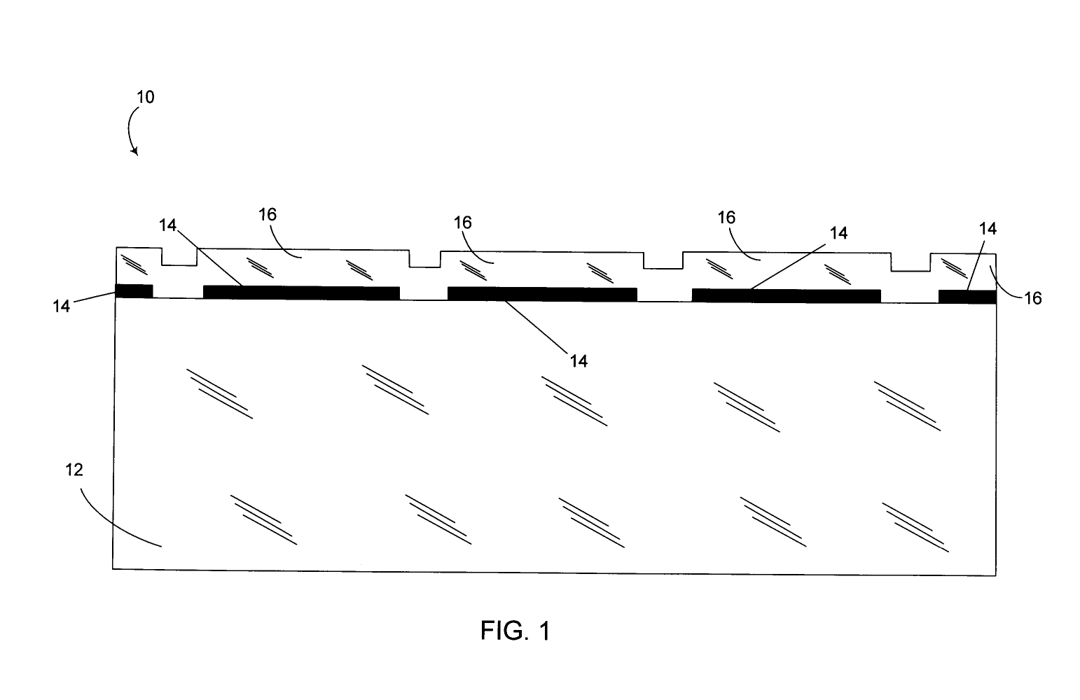

FIG. 1 is a cross-sectional diagrammatic view of a binary photomask. The photomask 10 includes a substantially transparent substrate 12 having an opaque pattern 14 formed thereon. The substrate 12 may partially or substantially include fused quartz (e.g., SiO₂), and/or calcium fluoride (CaF₂), and/or other materials or combinations thereof. The patterned layer 14 may include chromium, chromium alloy, iron oxide, or an inorganic film made with MoSi, ZrSiO, SiN, and/or TiN, among other materials. The pattern is formed in layer 14 using one of a number of conventional etching techniques.

The photomask 10 also includes a protective layer 16 that is formed directly on the opaque pattern 14 and substrate 12. The protective layer 16 is preferably transparent and has a thickness in the range of 300-800 Angstroms, e.g., 500 Å. In one example, the protective layer 16 includes silica (SiO₂); although, other materials may be used. As shown in FIG. 1, the protective layer 16 together with substrate 12 sealingly encloses the patterned surface 14 of the substrate 12.

This sealing closes the volume around contaminants formed on the substrate 12 and patterned layer 14 during fabrication of those layers. Thus, when the contaminants are exposed during the photolithography process, there is not a volume for vaporization/sublimation of the contaminants. As a result, a contaminant layer or haze is not formed on the patterned surface of the substrate 12 during exposure.

Moreover, any haze that may form as a result of UV exposure of residue that is formed on the protective layer can be removed with cleaning of the protective layer 16 without significantly affecting the durability or performance of patterned layer 14. In this regard, the patterned layer 14 is not contaminated or damaged by repeated cleanings of the photomask. The protective layer 16 is more durable to the caustic characteristics of the cleaning solutions generally used to clean photomasks, such as a solution of ammonium and sulfate, than the patterned layer 14. Thus, the lifespan of the photomask 10 is increased relative to a conventional photomask.

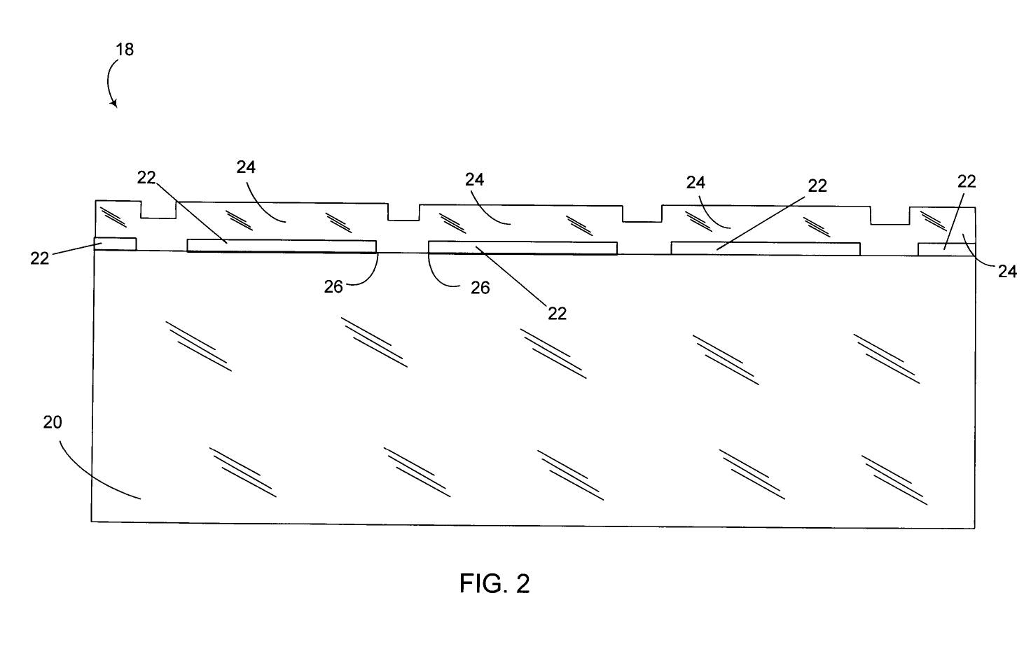

Referring now to FIG. 2, a cross-sectional diagrammatic view of an embedded attenuated phase shift photomask (PSM) 18 is shown. Photomask 18 is similar to the photomask 10 described with respect to FIG. 1 in that a quartz substrate 20 supports a patterned layer 22. Other materials may be used to form substrate 20, such as CaF₂ The pattern is formed in layer 22 using one of a number of conventional etching techniques. However, unlike the pattern layer of photomask 10 of FIG. 1, patterned layer 22 is formed of translucent rather than opaque material.

One exemplary translucent material is molybdenum silicide (MoSiOₓN_{y}). Unlike chrome or other opaque materials, molybdenum silicide and other translucent materials are designed to allow a small fraction of light pass therethrough during exposure of the photomask. The light that passes through the patterned portions, however, is not strong enough to expose the pattern on the wafer (not shown). The relatively weak light that does pass through the patterned portions is 180° out of phase with the light that passes through the unprotected portions of the photomask substrate 20. As such, where the translucent material and quartz substrate meet 26, the light interferes in such a manner to sharpen the edges of the translucent pattern. This phenomenon is often exploited for fabricating integrated circuits with ever shrinking linewidths, e.g., 0.13 micron.

Photomask also has a protective layer 24 formed over the patterned surface. The protective layer 24 is formed of transparent material, e.g., silica, and seals the patterned surface of the photomask. This sealing closes the volume around contaminants that may be present on the patterned substrate 20, 22. Thus, when the contaminants are exposed during the photolithography process, there is not a volume for vaporization/sublimation of the contaminants. As a result, a contaminant layer or haze is not formed on the patterned surface of the substrate 20 during exposure.

Moreover, any haze that may form as a result of UV exposure of residue that is formed on the protective layer can be removed in subsequent cleanings without significantly affecting the durability or performance of patterned layer 22.

The protective layers 16 and 24 may be formed by electroplating, electroless plating, spin-on coating, chemical vapor deposition (CVD), physical vapor deposition (PVD) such as evaporation and sputtering, or a combination thereof. The protective layers 16 and 24 may also be formed by implant or doping procedures. The protective layers and photomask substrates may be formed of the same or different materials, such as silica (SiO₂) or ice (H₂O) for exposure wavelengths greater than 193 nm, or calcium fluoride (CaF₂) for an exposure wavelength of 157 nm. One skilled in the art will appreciate that materials other than those specifically identified may be used. The photomasks 10 and 18 may also include one or more adhesive layers (not shown) to enhance the adhesion of the protective layers to the patterned layers and substrates.

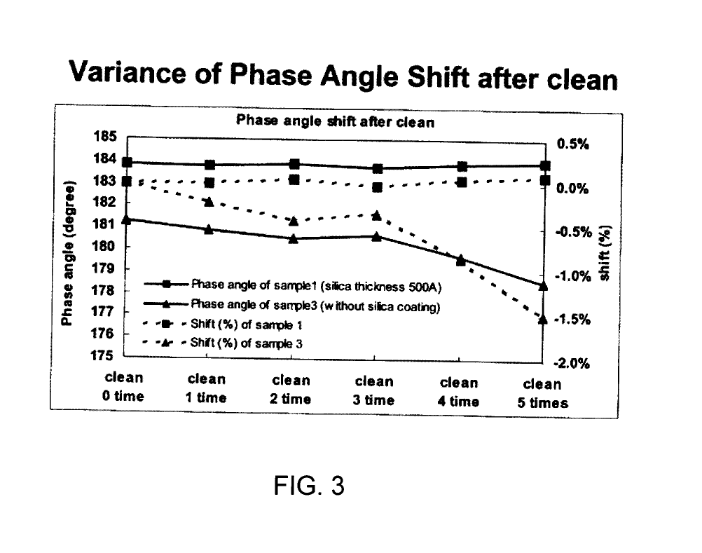

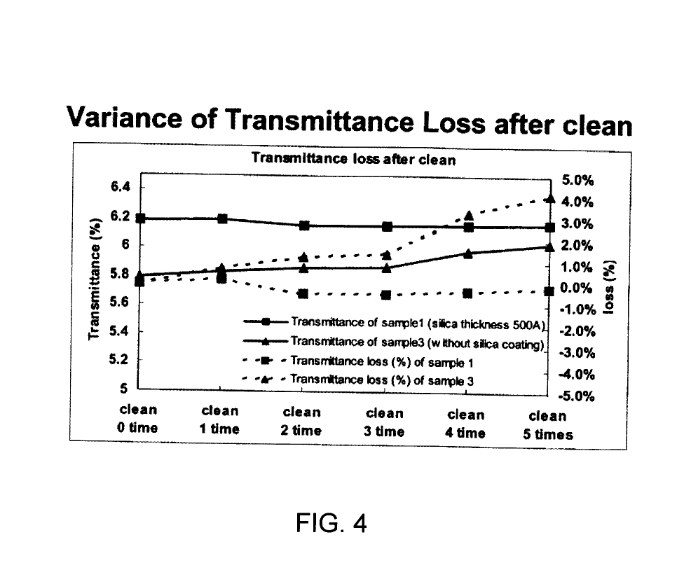

As described above, the durability of the photomask is enhanced by implementation of a protective passivation layer formed on the patterned photomask substrate. This passivation layer, not only improves photomask durability, but maintains photomask performance within performance tolerances despite numerous cleanings of the photomask. For example, a photomask according to an aspect of the present disclosure was repeatedly cleaned and exposed to deep ultraviolet light (DUV), and data regarding phase angle shift and transmittance was gathered and compared to that of a conventional mask. These results for two 248 nm phase shift masks (PSM) are shown in FIGS. 3-4.

FIG. 3 is a histogram showing variance of phase angle shift of a 248 nm PSM coated with a 500 Å silica protective layer (sample 1) compared to a conventional 248 PSM absent a protective layer (sample 3). As shown, after five cleanings, the variance in phase shift for the coated PSM (sample 1) is less than 0.1%. That is, prior to any cleaning, the phase angle of the coated PSM (sample 1) was approximately 184°. After the cleanings, the phase angle of the same PSM was still approximately 184°. Contrastingly, the phase angle of the non-coated PSM (sample 3) prior to any cleaning was approximately 181°. After the five cleanings, the phase angle for the non-coated PSM (sample 3) was found to be approximately 178°, which corresponds to a 1.5% phase shift variance. Thus, relative to the non-coated PSM, the PSM coated with a silica protective layer yielded a 15× reduction in phase angle variance.

FIG. 4 is a histogram showing variance of transmittance loss for the 248 nm PSM having a 500 Å silica protective layer (sample 1). The data shown in the figure compares the coated PSM to a non-coated 248 nm PSM (sample 3). As shown in the figure, the transmittance of the coated PSM prior to any cleaning was approximately 6.2%. After five cleanings, the transmittance was found to be slightly less than 6.2%, for a transmittance loss variance of less than 0.2%. On the other hand, the non-coated PSM (sample 3) had a transmittance of approximately 5.8% and after the five cleanings, the sample had a transmittance loss of approximately 6.0%. This amounts to variance in excess of 4.0%. Thus, the silica coating on the PSM (sample 1) provided a nearly 20 fold reduction in transmittance loss relative to the non-coated PSM (sample 3).

The present disclosure has been described with respect to a photomask fabricated to have a protective coating or layer that sealingly encloses, in a haze-prevention manner, contaminants present on a patterned surface of the photomask. The protective coating also prevents cleaning chemicals from impinging on the patterned surface of the photomask during cleaning of the photomask. A similar protective or coating layer may also be used with other semiconductor fabrication components. Additionally, the protective layer may be used to reduce electrostatic discharge induced defects. Moreover, application of a protective layer on a patterned surface of a photomask was found to have little impact on the critical dimension (CD) of the photomask. For example, a 193 nm PSM with a CD of 1.100 microns was found to have a CD of 1.104 microns after application of a protective layer.

It is recognized that equivalents, alternatives, and modifications, aside from those expressly stated, are possible and within the scope of the appending claims.

What is claimed is:

1. A method of fabricating a photomask, the method comprising:
providing a photomask substrate, the photomask substrate comprised of a first material;
patterning the photomask substrate to form a patterned surface; and
forming a barrier layer on the patterned surface, the barrier layer comprised of the first material.

2. The method of claim 1 wherein the forming of the barrier layer includes sealing chemical residue, formed during fabrication of the photomask, on the patterned surface of the photomask substrate.

3. The method of claim 1 wherein the forming of the barrier layer includes forming a silica layer on the patterned surface.

4. The method of claim 1 further comprising forming the photomask substrate to include MoSiOₓN_{y}.

5. The method of claim 1 wherein the barrier layer is formed by chemical vapor deposition.

6. The method of claim 1 further comprising forming the barrier layer to have a thickness between 300 and 800 Å.

7. The method of claim 6 further comprising forming the barrier layer to have a thickness of approximately 500 Å.

8. The method of claim 1 wherein the forming of the barrier layer includes spin-coating of the barrier layer on the patterned surface.

9. The method of claim 1 wherein the patterning includes depositing an opaque layer on the photomask substrate and etching the opaque layer.

10. The method of claim 1 wherein the patterning includes depositing a translucent layer on the photomask substrate and etching the translucent layer.

11. An apparatus comprising a photomask that includes:
a transparent layer;
a patterned layer formed on the transparent layer; and
a haze reduction layer formed on the patterned layer.

12. The apparatus of claim 11 wherein the haze reduction layer seals the patterned layer from cleaning materials used during cleaning of the photomask.

13. The apparatus of claim 11 wherein the haze reduction layer includes silica.

14. The apparatus of claim 11 wherein the haze reduction layer is deposited on the patterned layer by chemical vapor deposition.

15. The apparatus of claim 11 wherein the haze reduction layer has a thickness of approximately 500 Å.

16. The apparatus of claim 11 wherein the patterned layer has a critical dimension that is minimally affected by the haze reduction layer.

17. The apparatus of claim 11 wherein the haze reduction layer is spin-coated onto the patterned layer.

18. The apparatus of claim 11 wherein the patterned layer is opaque.

19. The apparatus of claim 18 wherein the patterned layer is formed of chrome.

20. The apparatus of claim 11 wherein the patterned layer is translucent.

21. The apparatus of claim 20 wherein the patterned layer is formed of molybdenum silicide.

22. The apparatus of claim 11 wherein the photomask is a binary photomask.

23. The apparatus of claim 11 wherein the photomask is a phase shift mask (PSM).

24. The apparatus of claim 11 wherein the first transparent layer and the haze reduction layer sealingly enclose the patterned layer.

25. The apparatus of claim 11 wherein the haze reduction layer is transparent.

26. The apparatus of claim 11 wherein the transparent layer and the haze reduction layer are each comprised of silica.

27. The apparatus of claim 11 wherein the transparent layer and the haze reduction layer are each comprised of quartz.
